(19) United States
(12) Patent Application Publication
(10) Pub. No.: US 2006/0058561 A1
(43) Pub. Date: Mar. 16, 2006

(54) Process for dehydrating 2-methylpentane-2,4-diol

(75) Inventor: Helmut Springer, Dinslaken (DE)

Correspondence Address: HEDMAN & COSTIGAN P.C., 1185 AVENUE OF THE AMERICAS, NEW YORK, NY, 10036, US

(73) Assignee: Celanese Chemicals Europe GmbH

(21) Appl. No.: 11/184,136

(22) Filed: Jul. 19, 2005

(30) Foreign Application Priority Data
Sep. 11, 2004 (DE) 10 2004 043 940.0

(51) Int. Cl. C07C 1/00

(52) U.S. Cl. 585/603

(57) ABSTRACT

A process for dehydrating 2-methylpentanediol-2,4 to a mixture of 2-methyl-1,3-pentadiene and 4-methyl-1,3-pentadiene at elevated temperature in the presence of an acid catalyst using a polyglycol ether as a heat carrier, wherein a polyglycol ether containing from 80 to 100% by weight of a polyethylene glycol dimethyl ether of the formula CH₃(OCH₂CH₂)ₙOCH₃ where n=2-8 and from 0 to 20% by weight of a polyethylene glycol monomethyl ether of the formula CH₃(OCH₂CH₂)ₙOH where n=2-8 based in each case on the total mass of polyglycol ether, is used.

A process for dehydrating 2-methylpentane-2,4-diol to a mixture of 2-methyl-1,3-pentadiene and 4-methyl-1,3-pentadiene under acidic catalysis in a polyglycol ether as a heat carrier.

### STATE OF THE ART

It is known that a mixture of 2-methyl-1,3-pentadiene and 4-methyl-1,3-pentadiene, referred to herein below as methylpentadiene, can be prepared by dehydrating 2-methylpentane-2,4-diol. As the starting material for the preparation of fragrances, methylpentadiene has particular significance. For example, a Diels-Alder reaction with suitable olefinically unsaturated compounds leads to substituted cyclohexene derivatives which have again gained high significance for the preparation of fragrance compositions.

The starting material 2-methylpentane-2,4-diol is obtainable in know processes by condensation of acetone to diacetone alcohol with subsequent hydrogenation according to GB 572,602. In GB 572,602, the dehydration is effected in the presence of iodine or hydrogen chloride. However, the use of these volatile dehydration catalysts presents problems; for example, corrosion problems on the plant parts are to be expected.

A further process for dehydrating 2-methylpentane-2,4-diol is known from JP-A-61-57525. In this process, the dehydration catalyst used is an acidic compound which is present dissolved in a particular glycol or in a particular glycol ether. 2-Methylpentane-2,4-diol is introduced continuosly into this mixture at elevated tempatures in a range from 90 to 200° C. Also disclosed is the batchwise reaction in which the catalyst-containing solvent is initially charged together with 2-methylpentane-2,4-diol and then heated. The glycols used are, for example, diethylene glycol or propylene glycol, and the glycol ethers used are diethylene glycol monobutyl ether, diethylene glycol dibutyl ether or tetraethylene glycol monomethyl ether.

The desired dehydration product and water of reaction is distilled out of the reaction system and purified after water removal. Suitable acidic catalysts are mineral acids or organic acids such as sulfuric acid, phosphoric acid or p-toluenesulfonic acid, and the acidic salts thereof. The known process is characterized by the use of a particular glycol or glycol ether. The advantageous action of the glycols as a solvent is indicated by their dissolution capability for high-boiling condensation products which can impair the activity of the acidic catalyst.

SU-A-1-1,759,825 likewise contemplates a process for dehydrating 2-methylpentane-2,4-diol using particular glycols, specifically ethylene glycol, diethylene glycol or triethylene glycol, as a high-boiling solvent. The catalytically active compound is prepared by dissolving potassium hydroxide in the glycol at a temperature of 20-85° C. and subsequently reacting with sulfuric acid. The actual dehydration reaction is carried out batchwise after addition of 2-methylpentane-2,4-diol and is effected in the presence of hydroquinone, the desired dehydration product being distilled out of the reaction mixture. Compared to the process from JP-A1-61-57525, the methylpentadiene yield and the proportion of the desired 2-methyl-1,3-pentadiene can be improved when a molar ratio of 2-methylpentane-2,4-diol:potassium hydroxide:sulfuric acid:glycol=1:(0.10-0.25):(0.10-0.25):(0.2-1.0) is observed, and when the glycol used is ethylene glycol, diethylene glycol or triethylene glycol. In the case of the batchwise dehydration, SU-A 1-1,759,825 reports an increase in yield of methypentadiene to 63-72% with a content of 2-methyl-1,3-pentadiene of 75 to 80%. SU-A1-1,759,825 likewise indicates that the use of ethylene glycol monophenyl ethers does not show any advantageous effect.

The prior art requires a costly and inconvenient preparation process for the acidic catalyst and the use of particular glycols in pure form as a solvent. An exact molar ratio between the reaction components likewise has to be observed in order to ensure a sufficient dehydration reacted.

### THE INVENTION

It has now been found that, surprisingly, 2-methylpentane-2,4-diol can be dehydrated in high yields to a mixture of 2-methyl-1,3-pentadiene and 4-methyl-1,3-pentadiene (methylpentadiene) when aqueous solutions of acidic salts of mineral acids are initially introduced into industrially available polyglycol ethers as heat carriers, and 2-methylpentane-2,4-diol is subsequently treated with the thus prepared solution at elevated temperature.

The invention therefore consists in a process for dehydrating 2-methylpentanediol-2,4 to a mixture of 2-methyl-1,3-pentadiene and 4-methyl-1,3-pentadiene in the presence of an acid catalyst using a polyglycol ether as a heat carrier, comprising introducing an aqueous solution of an acidic salt of a mineral acid into a polyglycol ether containing from 80 to 100% by weight of a polyethylene glycol dimethyl ether of the formula [CH₃(OCH₂CH₂)ₙOCH₃ where n=2-8] and from 0 to 20% by weight of a polyethylene glycol monomethyl ether of the formula [CH₃(OCH₂CH₂)ₙOH where n=2-8], based in each case on the total mass of polyglycol ether, and the remainder to 100% by weight consisting substantially of high boilers, and treating 2-methylpentane-2,4-diol with the thus prepared solution at elevated temperatures.

In the process according to the invention, it is possible in a simple manner to use aqueous solutions of commercially available acidic salts of mineral acids. Examples of such acidic salts are alkali metal or alkaline earth metal salts, such as potassium or sodium hydrogensulfate, potassium or sodium dihydrogenphosphate and dipotassium or disodium hydrogenphosphate. Preference is given to using the inexpensive available potassium hydrogensulfate. Typically, an aqueous solution having a content of from 5 to 25% by weight of acid salt is used.

In the process according to the invention, the high-boiling solvents, which are also referred to as heat carriers, are of particular significance. Surprisingly, particularly useful polyglycol ethers have been found to be industrially available polyglycol ethers in which different polyethylene glycol dimethyl ethers are present in a mixture, in some cases with a proportion of polyethylene glycol monomethyl ethers. In this context, mixtures containing from 80 to 100% by weight of a polyethylene glycol dimethyl ether of the formula [CH₃(OCH₂CH₂)ₙOCH₃ where n=2-8] and from 0 to 20% by weight of a polyethylene glycol monomethyl ether of the formula [CH₃(OCH₂CH₂)ₙOH where n=2-8], based in each case on the total mass of polyglycol ether, should be used. The remainder to 100% by weight consists substantially of high boilers. Particularly suitable are mixtures with polyethylene glycol dimethyl ethers in which the dimethyl ether content, normalized to 100%, is divided up as follows: not more that 65% by weight of triethylene glycol dimethyl ether (n=3), not more than 75% by weight of tetraethylene glycol dimethyl ether (n=4) and not more than 20% by weight of pentaethylene glycol dimethyl ether (n=5). The remainder to 100% by weight consists substantially of hexa-, hepat- and octaethylene glycol dimethyl ethers, and high boilers. Such products are commercially available, for example the products PEG-DME 180 and PEG-DME 200 from Clariant GmbH.

Surprisingly, the use of pure polyethylene glycol dimethyl ethers, for example of pure triethylene glycol dimethyl ether, as a high-boiling solvent or heat carrier leads to lower yields of methylpentadiene.

The dehydration of 2-methyl-2,4-pentanediol is appropriately carried out by a continuous process, wherein 2-methyl-2,4-pentanediol is introduced over time into the heated, catalyst-containing heat carrier, and the cleavage products and water of reaction are simultaneously distilled off. The batchwise procedure, in which the entire amount of 2-methyl-2,4-pentanediol is initially added to the heat carrier with the catalyst present therein, and in which the reaction mixture is subsequently heated and the cleavage products are distilled off, is not ruled out, but is the less preferred variant.

In the preferred continuous procedure, the heat carrier is initially charged and mixed with the aqueous solution of the acidic salt of the mineral acid. The mixture is heated to a temperature of from 110 to 200° C., in the course of which water which has been added and is present in the heat carrier is distilled off. The acidic salt precipitates out in finely crystalline form and can be suspended in a simple manner in the heat carrier, for example by stirring. Subsequently, the temperature in the heat carrier is adjusted to 120 to 200° C., preferably to 160 to 190° C., and 2-methyl-2,4-pentanediol is introduced at a constant rate. At the same time as 2-methyl-2,4-pentanediol is introduced into the heat carrier, the cleavage products are distilled out of the heat carrier together with the water formed. The dehydration of 2-methyl-2,4-pentanediol is effected generally under standard pressure, but the use of reduced pressures is not ruled out, in order, for example, to more rapidly remove methylpentadiene from the hot reaction zone.

The cleavage products which have been distilled off are collected in a receiver and separated from the water which separates off. Subsequently, methylpentadiene is removed distillatively from the organic crude product.

The quantitive ratio between the initially charged heat carrier and the added 2-methyl-2,4-pentanediol, and the feed rate of 2-methyl-2,4-pentanediol if the continuous process is employed, may be varied over a wide range and is not critical for the performance of the process according to the invention. It depends upon the apparatus circumstances and can be optimized by routine studies.

The amount of catalyst suspended in heat carrier may also be varied over a wide range and can be determined by routine experiments. Excessively small amounts of catalyst do not bring about sufficient cleavage and excessively large amounts of catalyst no longer exhibit any positive effect, but rather unnecessarily make the process more expensive. In general, from 5 to 20, preferably from 10 to 15, parts by weight of acid catalyst, calculated as pure substance, are used in the form of an aqueous solution per 100 parts by weight of heat carrier.

The process according to the invention gives rise in a simple manner to organic crude products having a content methylpentadiene (2-methyl-1,3-pentadiene and 4-methyl-1,3-pentadiene) of 65-70% by weight. Based on the used amount of 2-methyl-2,4-pentanediol, the yield of methylpentadiene detected by distillation is between 58-63%.

In contrast to the process known from the prior art SU-A1-1 759 825, the claimed process is notable for a particularly simple operating mode. The catalyst need not first be prepared in a separate stage, but rather it may be used directly as an aqueous solution starting from commercially available compounds. The complex establishment of the complicated molar ratio of 2-methylpentane-2,4-diol:potassium hydroxide:sulfuric acid:glycol of 1:(0.10-0.25):(0.10-0.25): (0.2-1.0) is also not necessary in the dehydration process according to the invention.

It is found that, surprisingly, the yield of methylpentadiene depends upon the heat carrier used. While low yields are observed when pure polyglycol ethers are used, it is possible to increase the methylpentadiene yield when polyglycol ethers are used in technical-grade quality, which are in the form of mixtures.

In the following examples, there are described several preferred embodiments to illustrate the invention. However, it should be understood that the invention is not intended to be limited to the specific embodiments.

### EXAMPLES

### 1. Dehydration of 2-methyl-2,4-pentanediol

The apparatus for liquid phase dehydration of 2-methyl-2,4-pentanediol consisted of a 500 ml round-bottom flask equipped with a stirrer, internal thermometer and a 30 cm Vigreux column with Claisen bridge and receiver.

The 500 ml round-bottom flask was initially charged with 300.0 g of technical-grade triethylene glycol dimethyl ether (PEG-DME 180 from Clariant GmbH) with 53% by weight of triethylene glycol dimethyl ether, 26% by weight of tetraethylene glycol dimethyl ether, 8% by weight of pentaethylene glycol dimethyl ether and 13% by weight of remainder (predominantly monomethyl ethers) as the heat carrier, and the mixture was admixed with an aqueous solution of 40.8 g of potassium hydrogensulfate in 80.0 g of water. The mixture was heated to 180° C. with stirring, in the course of which the water used distilled off virtually quantitatively. As this was done, the potassium hydrogensulfate precipitated out in finely crystalline form and was suspended in the heat carrier with stirring. 80.0 g of 2-methyl-2,4-pentanediol per hour were pumped into the suspension. At a top temperature of 89° C., the cleavage products which formed distilled off and were collected in a receiver. After the water of reaction had been removed, an organic crude product of the following composition was obtained:

gas chromatography analysis (in area %):

| | |
|---|---|
| first runnings | 0.7 |
| isobutene | 7.5 |
| 2-methyl-1,3-pentadiene* | 69.6 |
| 2-methylpent-2-en-4-ol | 9.1 |
| others | 13.1 |

*including 4-methyl-1,3-pentadiene

The distillative workup of the crude product on a 24-tray column at standard pressure gave the following result:

TABLE 1
Distillative workup of crude methylpentadiene

| | Temperature | | | | |
|---|---|---|---|---|---|
| | (° C.) | | | Weight (% by weight) | |
| Fraction | Top | Bottom | Reflux ratio | Org. phase | Water phase |
| Cold trap | | | | 9.2 | |
| 1 | 65 | 84 | 5:1 | 3.9 | 0.2 |
| 2 | 67 | 85 | 5:1 | 1.5 | 0.1 |
| 3 | 80 | 135 | 2:1 | 56.0 | 1.1 |
| 4 | 122 | 180 | 1:1 | 21.8 | 0.5 |
| Residue | | | | 5.7 | |

TABLE 2
Gas chromatography analysis (in area %) of the fractions obtained by distillation

| Fraction | Cold trap | 1 | 2 | 3 | 4 | Residue |
|---|---|---|---|---|---|---|
| First runnings | | 0.89 | 0.30 | 0.01 | 0.04 | |
| Isobutene | 100.0 | 11.71 | 4.87 | 0.14 | 0.66 | |
| 2-Methyl-1,3-pentadiene* | | 76.81 | 85.26 | 97.82 | 6.15 | not determined |
| 2-Methylpent-2-en-4-ol | | | | 0.01 | 40.96 | |
| Others | | 10.59 | 9.57 | 2.02 | 52.19 | |
| Sulfur content: | | <0.2 ppm | 0.3 ppm | <0.2 ppm | 12 ppm | |

*including 4-methyl-1,3-pentadiene

### 2. Comparative Example

The procedure was analogous to Example 1 with the single exception that the heat carrier used was pure triethylene glycol dimethyl ether (content 99% by weight) instead of technical-grade triethylene glycol dimethyl ether.

According to gas chromatography analysis (data in area %), the resulting organic cleavage product had a content of 2-methyl-1,3-pentadiene/4-methy-1,3-pentadiene of 56% and the content of 2-methylpent-2-en-4-ol was 26%. As the inventive example and the comparative example demonstrate, the nature of the heat carrier has a significant influence on the degree of dehydration of 2-methyl-2,4-pentanediol. Various modifications of the process of the invention may be made without departing from the spirit or scope thereof and it is to be understood that the invention is intended to be limited only as defined in the appended claims.

What is claimed is:

1. A process for dehydrating 2-methylpentanediol-2,4 to a mixture of 2-methyl-1,3-pentadiene and 4-methyl-1,3-pentadiene in the presence of an acid catalyst using a polyglycol ether as a heat carrier, comprising introducing an aqueous solution of an acidic salt of a mineral acid into a polyglycol ether containing from 80 to 100% by weight of a polyethylene glycol dimethyl ether of the formula CH₃(OCH₂CH₂)ₙOCH₃ where n=2-8 and from 0 to 20% by weight of a polyethylene glycol monomethyl ether of the formula CH₃(OCH₂CH₂)ₙOH where n=2-8, based in each case on the total mass of polyglycol ether, and the reminder to 100% by weight consisting substantially of high boilers, and treating 2-methylpentane-2,4-diol with the thus prepared solution at elevated temperature.

2. The process of claim 1, wherein the polyethylene glycol dimethyl ether, normalized to 100%, contains not more than 65% by weight of triethylene glycol dimethyl ether, not more than 75% by weight of tetraethylene glycol dimethyl ether and not more than 20% by weight of pentaethylene glycol dimethyl ether, and the remainder to 100% by weight consists substantially of hexa-, hepta- and octaethylene glycol dimethyl ethers, and also high boilers.

3. The process of claim 1, wherein an aqueous solution of an acidic salt of a material acid is introduced into the polyglycol ether, the polyglycol ether is first heated to 110 to 200° C. and water present is distilled off, then the temperature is adjusted to 120° to 200° C. after which, 2-methylpentanediol-2,4 is added and the dehydration products are simultaneously distilled off from the polyglycol ether.

4. The process of claim 3, wherein the temperature is adjusted to 160 to 190° C. after the water has been distilled off.

5. The process of claim 1, wherein the acidic salts of a mineral acid used are alkali metal or alkaline earth metal salts.

6. The process of claim 5, wherein the acidic salts used are selected from the group consisting of potassium hydrogensulfate, sodium hydrogensulfate, potassium dihydrogenphosphate, dipotassium hydrogenphosphate, sodium dihydrogenphosphate and disodium hydrogenphosphate.

7. The process of claim 1, wherein the aqueous solution of the acidic salt of a mineral acid has a content of from 5 to 25% by weight of acidic salt.

8. The process of claim 1, wherein from 5 to 20 parts by weight of acid catalyst, calculated as pure substance, are added in the form of an aqueous solution per 100 parts by weight of heat carrier.

9. The process of claim 8 where 10 to 15 parts by weight of acid catalyst are used.
